(19) United States
(12) Patent Application Publication
(10) Pub. No.: US 2013/0202703 A1
(43) Pub. Date: Aug. 8, 2013

(54) METHOD FOR PRODUCING GARDENIA BLUE PIGMENT

(71) Applicant: Shin Sadano, Kyoto (JP)

(72) Inventor: Shin Sadano, Kyoto (JP)

(21) Appl. No.: 13/822,742

(22) PCT Filed: Sep. 7, 2011
(86) PCT No.: PCT/JP2011/070340

§ 371 Date: Apr. 22, 2013

(30) Foreign Application Priority Data
Sep. 27, 2010 (JP) 2010-214854

(51) Int. Cl. C09B 67/54; A23L 1/275; A61K 9/28

(52) U.S. Cl. CPC C09B 67/0096; A61K 9/2826; A23L 1/2751
USPC 424/479; 549/396; 426/103

(57) ABSTRACT

Provided is a method for producing a gardenia blue pigment resistant to discoloration which may occur in colored sugar-coated food or pharmaceutical products. The method for producing such a gardenia blue pigment comprises performing membrane separation using a membrane (for example, an ultrafiltration membrane) with a molecular weight cut-off of 3000 Da or larger for removal of low-molecular compounds from a solution resulting from β-glucosidase treatment of an iridoid glycoside (for example, geniposide etc.) in the presence of a protein hydrolysate (for example, a casein protein hydrolysate), the iridoid glycoside being obtainable by extraction from fruits of Gardenia jasminoides (Rubiaceae). Also provided is a sugar-coated food or pharmaceutical product (for example, a sugar-coated tablet, a sugar-coated chewing gum, etc.) having a sugar-coating layer colored with the gardenia blue pigment obtainable by the above-described method.

### TECHNICAL FIELD

The present invention relates to a method for producing gardenia blue pigment.

### BACKGROUND ART

To date, gardenia blue pigment has been widely used as a blue colorant for food. Generally, gardenia blue pigment is produced by enzymatic treatment of iridoid glycosides present in gardenia fruit extracts etc, with β-glucosidase in the presence of protein degradation products.

Known technologies regarding gardenia blue pigment include the following: a method for producing a brightened blue natural pigment from a gardenia iridoid glycoside with use of β-glucosidase, the method comprising treating a gardenia iridoid glycoside or a substance containing the same with β-glucosidase or a substance containing the same in the presence of a primary amino group-containing substance under aerobic conditions, the method being characterized in that the glycoside or the substance containing the same is sufficiently treated with β-glucosidase or the substance containing the same under microaerobic conditions beforehand, and then further treated similarly under stirring (see Patent Literature 1); and a gardenia blue pigment preparation having an improved color tone, the preparation being obtainable by extracting an iridoid glycoside from fruits of Gardenia jasminoides (Rubiaceae), treating the iridoid glycoside with β-glucosidase in the presence of a soybean protein degradation product (in the absence of taurine-containing substances) for preparation of gardenia blue pigment, and blending the gardenia blue pigment with an enzymatically modified isoquercitrin (see Patent Literature 2).

In the production of food or pharmaceutical products, for alleviation of quality loss resulting from moisture absorption, oxidation, photolysis, etc., or for reduction of bitterness, smell, irritation, etc., it is common to coat, with a sugar layer, the surface of a core material containing a food or pharmaceutical product (that is, sugar-coating). For blue coloring of such food products etc., gardenia blue pigment is blended into the sugar-coating layer in some cases. The thus-obtained colored food products etc. are of a bright blue color immediately after production, but easily turn dull blue during storage. Therefore, such a discoloration problem has been eagerly desired to be solved.

### CITATION LIST

### Patent Literature

Patent Literature 1: IT-A 56-92792
Patent Literature 2: Japanese Patent No. 4374494

### SUMMARY OF INVENTION

### Technical Problem

An object of the present invention is to provide a method for producing a gardenia blue pigment resistant to discoloration which may occur in colored sugar-coated food or pharmaceutical products.

### Solution To Problem

The present inventor conducted extensive research to solve the above-mentioned problem. As a result, the inventor found that separation and removal of low-molecular compounds, using a membrane with a predetermined molecular weight cut-off, from a solution resulting from β-glucosidase treatment of an iridoid glycoside in the presence of a protein hydrolysate can solve the above-mentioned problem, and then completed the present invention.

That is, the present invention comprises the following.
(1) A method for producing gardenia blue pigment, comprising performing membrane separation using a membrane with a molecular weight cut-off of 3000 Da or larger for removal of low-molecular compounds from a solution resulting from β-glucosidase treatment of an iridoid glycoside in the presence of a protein hydrolysate.
(2) The method according to the above (1), wherein the membrane separation is performed by ultrafiltration.
(3) A sugar-coated food or pharmaceutical product having a sugar-coating layer colored with the gardenia blue pigment obtainable by the method of the above (1) or (2).
(4) The food or pharmaceutical product according to the above (3), wherein the sugar-coating layer contains any one or more ingredients selected from sucrose, glucose, palatinose, reduced palatinose, reduced lactose, erythritol, xylitol, maltitol and mannitol.

### Advantageous Effects of Invention

Sugar-coated tablets colored with the gardenia blue pigment obtainable by the production method of the present invention are resistant to pigment discoloration during storage.

### DESCRIPTION OF EMBODIMENTS

The gardenia blue pigment of the present invention is produced by performing membrane separation using a membrane with a molecular weight cut-off (MWCO) of 3000 Da or larger for removal of low-molecular compounds from a solution resulting from β-glucosidase treatment of an iridoid glycoside in the presence of a protein hydrolysate.

The iridoid glycoside used for the present invention is not particularly limited as long as the iridoid glycoside is obtainable by extraction from fruits of Gardenia jasminoides (Gardenia augusta MERRIL var. grandiflora HORT. or Gardenia jasminoides ELLIS), a member of the family Rubiaceae. For example, geniposide is preferably used.

There is no limitation on the method for extracting geniposide from the above-mentioned gardenia fruits. For example, the following known method can be used: dried gardenia fruits are ground and then geniposide is extracted therefrom with water, an alcohol or a mixed solvent thereof. As for the extraction conditions, for example in the case where a mixed solvent of water and an alcohol (1:1) is used, the temperature and duration are preferably room temperature (about 0 to 30° C.) to 50° C. and about 1 to 18 hours, and more preferably about 30 to 40° C. and about 2 to 4 hours. For increase in extraction ratio of geniposide from the ground dried fruits, this extraction procedure is usually repeated more than once. The geniposide-containing extract is concentrated according to a method known per se, and the concentrated liquid is usually refrigerated or frozen for storage.

The concentrated liquid is usually treated with an adsorbent for removal of ingredients except geniposide, including the yellow pigment crocin and other ingredients. For example, treatment with an adsorption resin is performed as follows.

First, the concentrated liquid is diluted to a suitable concentration, and the diluted solution is applied to a column filled up with an adsorption resin. Examples of the adsorption resin include porous resins such as Amberlite XAD-4 and Amberlite XAD-7 (product names; manufactured, by ORGANO CORPORATION), DIAION HP-20, HP-21 and HP-40 (product names; manufactured by Mitsubishi Chemical Corporation), and the like. Amberlite XAD-7 is preferably used.

Next, water or a mixed solvent of a low-concentration alcohol (for example, ethanol etc.) and water is passed through the column, and the unadsorbed and eluted fraction is collected. Thus, the geniposide-containing fraction can be obtained. This fraction is concentrated according to a method known per se, and the concentrated liquid is usually refrigerated or frozen for storage.

The protein hydrolysate used for the present invention is, for example, a hydrolysate of proteins derived from plants such as soybeans and wheat, a hydrolysate of animal-derived proteins such as casein and gelatin, or a hydrolysate of proteins derived from microorganisms such as yeasts. Examples of the protein hydrolysate used for the present invention include ones commercially produced and sold, such as HI-NUTE R (trade name; manufactured by FUJI OIL Co., Ltd.; derived from soybeans), EPS-C (trade name; manufactured by BANSHU CHOMIRYO Co., Ltd.; derived from corns), Pro Ekisu G2 (trade name; manufactured by BANSHU CHOMIRYO Co., Ltd.; derived from corns), CPOP (trade name; manufactured by Morinaga Milk Industry Co., Ltd.; derived from casein), CU2500 (tradename; manufactured by Morinaga Milk Industry Co., Ltd.; derived from casein), Lactaminosan (trade name; manufactured by Cosmo Foods Co., Ltd.; derived from casein), FCP-A. (trade name; manufactured by Nippi, Inc.; derived from gelatin), Yeast Extract FR (trade name; manufactured by Kirin Food-Tech Company, Limited; derived from yeasts), and Yeast Extract SL-W (trade name; manufactured by Kirin Food-Tech Company, Limited; derived from yeasts). These can be used in the present invention.

The β-glucosidase used for the present invention is not particularly limited as long as it has β-glucosidase activity. For example, β-glucosidases derived from Aspergillus niger, Trichoderma reesei, Trichoderma viride, an almond and the like are included. Examples of the β-glucosidase used for the present invention include ones commercially produced and sold, such as Sumizyme C6000, Sumizyme AC, Sumizyme C, Sumizyme X, Sumizyme BGT and Sumizyme BGA (trade names; manufactured by SHINNIHON CHEMICALS Corporation); Cellulosin AC40, Cellulosin T3 and Cellulosin AL (trade names; manufactured by HBI Enzymes Inc.); ONOZUKA 3S and Y-NC (trade names; manufactured by Yakult Pharmaceutical Industry Co., Ltd.); and Cellulase A “Amano” 3 and Cellulase T “Amano” 4 (trade names; manufactured by Amano Enzyme Inc.). These can be used in the present invention.

The β-glucosidase treatment can be performed according to any method without particular limitation as long as the method can produce gardenia blue pigment. For example, the β-glucosidase treatment can be performed by mixing an iridoid glycoside, a protein hydrolysate and water into an aqueous solution, adding β-glucosidase thereto, and stirring or shaking the resulting mixture.

The conditions for the above-mentioned treatment can be as follows: the temperature is usually about 20 to 70° C., and preferably about 40 to 60° C.; the pH is usually 4 to 6, and preferably 4.5 to 5.5; and the reaction duration is usually about 30 minutes to 50 hours, and preferably about 15 to 30 hours.

For adjustment of the pH in the above-mentioned treatment, it is preferable to add a suitable amount of an alkaline agent (for example, sodium hydroxide, potassium hydroxide, sodium carbonate, potassium carbonate, etc.) to the aqueous solution before addition of β-glucosidase.

The above-mentioned treatment is preferably performed under aerobic conditions. Maintaining the aerobic conditions can be achieved, for example by mechanical means such as stirring and shaking, or by injection of a molecular oxygen-containing gas such as air.

β-glucosidase may be added in any manner without particular limitation. For example, β-glucosidase may be directly added at once. Alternatively, an aqueous solution of β-glucosidase may be prepared and then added at once or in 2 to 50 divided portions.

The aqueous solution in the above-mentioned treatment is preferably adjusted so as to contain about 1 to 20 mass %, preferably about 4 to 15 mass % of the iridoid glycoside; and about 1 to 25 mass %, preferably about 4 to 15 mass % of the protein hydrolysate relative to 100 mass % of the aqueous solution. The amount of β-glucosidase added to the aqueous solution is preferably 0.01 to 1.0 g relative to 1 g of the iridoid glycoside.

The solution resulting from the above-mentioned treatment is heated for enzyme inactivation at about 70 to 100° C., preferably about 80 to 95° C., for about 10 minutes to 3 hours, preferably for about 10 minutes to 2 hours; diluted about 2- to 20-fold with water; and subjected to membrane separation using a membrane with MWCO of 3000 Da or larger. Using a membrane with MWCO of 3000 Da or larger allows efficient removal of low-molecular compounds such as glucose, amino acids and peptides. Since a membrane with MWCO exceeding 150000 eliminates the pigment ingredient in addition to low-molecular compounds, the maximum limit of the MWCO is usually about 150000.

The material Of the membrane used for the membrane separation may be natural or synthetic, for example, cellulose, cellulose diacetate, cellulose triacetate, polyamide, polysulfone, polystyrene, polyimide, polyacrylonitrile or the like.

The membrane separation can be performed by filtration using a functional polymer membrane, such as an ultrafiltration (UF) membrane and a reverse osmosis membrane (nano-filtration membrane). Industrially preferred is ultrafiltration.

Examples of the ultrafiltration membrane include ones commercially produced and sold, such as Microza SEP-3013 (trade name; manufactured by Asahi Kasei Chemicals Corporation; 3000 Da MWCO), Microza SIP-0013 (trade name; manufactured by Asahi Kasei Chemicals Corporation; 6000 Da MWCO), Microza SLP-3053 (trade name; manufactured by Asahi Kasei Chemicals Corporation; 10000 Da MWCO), Microza AHP-3013 (trade name; manufactured by Asahi Kasei Chemicals Corporation; 50000 Da MWCO), FUY03A1 (trade name; manufactured by Daicen Membrane-Systems Ltd.; 30000 Da MWCO) and FUS1582 (trade name; manufactured by Daicen Membrane-Systems Ltd.; 150000 Da MWCO). These can be used in the present invention.

The thus-obtained gardenia blue pigment in the form of an aqueous solution can be provided as it is as a pigment preparation. Alternatively, such a pigment preparation may be dried according to a method known per se and provided as a powdered pigment preparation. Examples of the drying method include vacuum-freeze drying, circulation drying, spray drying, vacuum drying, belt drying, shelf drying and drum drying. Preferred is vacuum-freeze drying. The loss on drying of the powdered pigment preparation is usually about 5 mass % or lower, and preferably about 1 to 3 mass %.

The gardenia blue pigment obtainable according to the present invention can be used for coloring of food or pharmaceutical products. There is no particular limitation on the food products to be colored, and examples thereof include frozen desserts such as ice cream containing a minimum of 8% w/w milk fat and 15% w/w total milk solids (ice cream in Japanese), ice cream containing a minimum of 3% w/w milk fat and 10% w/w total milk solids (ice milk in Japanese), ice cream containing a minimum of 3% w/w milk solids (lacto ice in Japanese), sherbet and other non-dairy frozen desserts; drinks such as a milk drink, a lactic acid bacteria drink, a soft drink, a carbonated drink, a fruit juice drink, a vegetable juice drink, a sport drink, a powdered drink, an alcoholic drink, a coffee drink and a tea drink; desserts such as pudding, jelly and yogurt; confectioneries such as chewing gums, chocolates, drops, hard candies, cookies, rice crackers and gummy candies; jams; soups; pickles; seasonings such as dressing and sauce; processed meat products such as hams and sausages; and fish jelly products such as fish sausages and fish cakes. There is no particular limitation on the pharmaceutical products to be colored, either, and examples thereof include antipyretic analgesics, antihistamines, antiallergics, sympathomimetics, parasympatholytics, central nervous system stimulants, H2 blockers, antacids, anti-inflammatory enzyme preparations, anti-inflammatory agents, bronchodilators, antibacterial agents, antitussives, expectorants, anticholinergics, antidiarrheals, sedative-hypnotics, cholagogues, antihypertensives, skeletal muscle relaxants, preventive and therapeutic drugs for motion sickness, vitamins and herbal medicines.

Further, since the gardenia blue pigment obtainable according to the present invention is excellent in resistance to discoloration which may occur in colored sugar-coated food or pharmaceutical products (for example, sugar-coated tablets, sugar-coated chewing gums, etc.), the gardenia blue pigment can be blended into the sugar-coating layer of such food or pharmaceutical products according to a method known per se and preferably used for such an application.

As an ingredient of the sugar-coating layer, any one or more selected from sucrose, glucose, palatinose, reduced palatinose, reduced lactose, erythritol, xylitol, maltitol and mannitol are preferably used.

### EXAMPLES

Hereinafter, the present invention will be illustrated in more detail by Production Examples and Working Examples, but is not limited thereto.

### Production Example

### Preparation of Geniposide Solution

To 1800 g of dried gardenia fruits (ground product), 7200 ml of a mixed solvent containing 40 vol % ethanol and water was added, and the mixture was stirred at room temperature for 3 hours and then subjected to suction filtration. Next, the following procedure was repeated twice: to the extraction residue, 3300 ml of a mixed solvent containing 40 vol % ethanol and water was added, and the mixture was stirred at room temperature for 30 minutes and then subjected to suction filtration. In total, 10500 ml of the filtrate was obtained as an extract solution. This extract solution was concentrated at 60° C. and 4 kPa with use of a rotary evaporator, and thus about 500 ml of the concentrated liquid, which contained geniposide, was obtained.

To the concentrated liquid, water was added to make a total volume of 1000 ml, and the diluted solution was loaded onto a column filled up with 3000 ml of Amberlite XAD-7 (product name; manufactured by ORGANO CORPORATION) at a flow rate of SV=0.5. Then, 24000 ml of water was passed through the column at a flow rate of SV=0.5, and the eluate was collected. The collected solution was concentrated at 60° C, and 4 kPa with use of a rotary evaporator, and thus 90 g of the concentrated liquid, which contained 47.1% of geniposide, was obtained.

### Working Example 1

### (1) Production of Gardenia Blue Pigment

An aqueous solution prepared by mixing 9.14 g of the concentrated liquid obtained in Production Example, 6.9 g of a soybean protein hydrolysate (trade name: HI-NUTE R; manufactured by FUJI OIL Co., Ltd.) and 33.4 g of water was adjusted to a pH of 5.5 with sodium hydroxide. To the aqueous solution, 0.3 g of β-glucosidase (trade name: Sumizyme C6000; manufactured by SHINNIHON CHEMICALS Corporation) was added and β-glucosidase treatment was performed under stirring at 50° C. for 24 hours. Heating at 90° C. was performed for 15 minutes to inactivate the enzyme and then insoluble substances were filtered off. Thus, 50 g of the reaction solution was obtained.

Fifty grams of the reaction solution was diluted with 250 g of water, and the diluted solution was applied to a 3000-Da MWCO ultrafiltration membrane (trade name: Microza SEP-0013; manufactured by Asahi Kasei Chemicals Corporation). Ultrafiltration was conducted at 0.7 kg/cm² and 35° C. Thus, the permeate fraction containing low-molecular substances was removed. The retentate fraction was subjected to pre-freezing in a freeze dryer (type: DC500; manufactured by Yamato Scientific Co., Ltd.) at −30° C. for 24 hours, and then freeze-dried at a vacuum degree of 10 Pa at a shelf temperature of 30° C. for about 70 to 72 hours. The freeze-dried product was ground in a pin mill with a 0.5-mm screen, and thus 7.0 g of a powdered gardenia blue pigment was obtained. The loss on drying of the gardenia blue pigment was 2.5 mass %.

### (2) Production of Colored Sugar-Coated Tablets

In 32 g of water, 0.02 g of the above-produced gardenia blue pigment and 68 g of maltitol (trade name: Lesys; manufactured by Mitsubishi Shoji Foodtech Co., Ltd.) were dissolved to give about 100 g of a coating liquid (solution temperature: 50° C.). Next, as a tablet core, 300 g of mock tablets containing lactose (average diameter: 8 mm) were charged into a pan-type granulator (model: PZ-01R; manufactured by AS ONE Corporation). For coating of the tablet cores, 4.0 g of the coating liquid was poured at once into the small sugar-coater continuously rotating at 25 rpm, and the tablet core surfaces were dried by an air flow intermittently fed into the small sugar-coater. This coating and drying procedure was repeated until the sugar-coating ratio reached 50%. Thus, sugar-coated tablets colored with gardenia blue pigment (Working Example Product 1) were obtained.

### Working Example 2

The same procedures as described in Working Example 1 were performed to give sugar-coated tablets colored with gardenia blue pigment (Working Example Product 2) , except that a 6000-Da MWCO ultrafiltration membrane (trade name: Microza SIP-0013; manufactured by Asahi Kasei Chemicals Corporation) was used instead of the 3000-Da MWCO ultrafiltration membrane (trade name: Microza SEP-0013; manufactured by Asahi Kasei Chemicals Corporation).

### Comparative Example 1

The same procedures as described in Working Example 1 (2) were performed to give sugar-coated tablets colored with gardenia blue pigment (Comparative Example Product 1), except that 0.13 g of the reaction solution prepared by the method of Working Example 1 (1) was used instead of 0.02 g of the gardenia blue pigment.

### Comparative Example 2

Five grams of the reaction solution prepared by the method of Working Example 1 (1) was diluted with 100 g of water, and the diluted solution was applied to a reverse osmosis membrane (trade name: NTR-7410; salt rejection rate: 10%; manufactured by NITTO DENKO CORPORATION). Reverse osmosis was conducted at 20 kg/cm² and 25° C. Thus, the permeate fraction containing low-molecular substances was removed. The retentate fraction was subjected to pre-freezing in a freeze dryer (type: DC500; manufactured by Yamato Scientific Co., Ltd.) at −30° C. for 24 hours, and then freeze-dried at a vacuum degree of 10 Pa at a shelf temperature of 30° C. for about 70 to 72 hours. The freeze-dried product was ground in a pin mill with a 0.5-mm screen, and thus 1.3 g of a powdered gardenia blue pigment was obtained. The loss on drying of the gardenia blue pigment was 2.0 mass %. Then, the same procedures as described in Working Example 1 (2) were performed to give sugar-coated tablets colored with gardenia blue pigment (Comparative Example Product 2), except that 0.03 g of the gardenia blue pigment obtained in this Example was used instead of 0.02 g of the gardenia blue pigment obtained in Working Example 1 (1).

### Evaluation

The sugar-coated tablets obtained in Working Examples and Comparative Examples (Working Example Products 1 and 2 and Comparative Example Products 1 and 2) were visually evaluated in terms of the color tone of the sugar-coated tablets immediately after production and after storage. As for the storage, the sugar-coated tablets obtained in each Example were put and sealed in a hermetic bag (made of polyethylene/aluminum/ethylene-methacrylic acid copolymer resin), and stored at 40° C. for one year. The results are shown in Table 1.

TABLE 1

| | Immediately after | |
|---|---|---|
| Sugar-coated tablet | production | After storage |
| Working Example | Bright blue | Bright blue |
| Product 1 | | |
| Working Example | Bright blue | Bright blue |
| Product 2 | | |
| Comparative Example | Bright blue | Dull blue |
| Product 1 | | |
| Comparative Example | Bright blue | Dull blue |
| Product 2 | | |

The results shown in Table 1 clearly demonstrate that the sugar-coated tablets colored with the gardenia blue pigment produced by the production method of the present invention are resistant to pigment discoloration as compared with those of Comparative Examples.

1. A method for producing gardenia blue pigment, comprising performing membrane separation using a membrane with a molecular weight cut-off of 3000 Da or larger for removal of low-molecular compounds from a solution resulting from β-glucosidase treatment of an iridoid glycoside in the presence of a protein hydrolysate.

2. The method according to claim 1, wherein the membrane separation is performed by ultrafiltration.

3. A sugar-coated food or pharmaceutical product having a sugar-coating layer colored with the gardenia blue pigment obtainable by the method of claim 1.

4. The food or pharmaceutical product according to claim 3, wherein the sugar-coating layer contains any one or more ingredients selected from sucrose, glucose, palatinose, reduced palatinose, reduced lactose, erythritol, xylitol, maltitol and mannitol.

5. A sugar-coated food or pharmaceutical product having a sugar-coating layer colored with the gardenia blue pigment obtainable by the method of claim 2.

6. The food or pharmaceutical product according to claim 5, wherein the sugar-coating layer contains any one or more ingredients selected from sucrose, glucose, palatinose, reduced palatinose, reduced lactose, erythritol, xylitol, maltitol and mannitol.
